SEQUENCE LISTING (14 sequences)

SEQ ID NO: 1 - protein, 269 aa, Mus musculus
MLLALLPVLGIHFVLRDAQAQSVTQPDARVTVSEGASLQLRCKYSYSGTPYLFWYVQYPRQGLQLLLKYYSGDPVVQGVNGFEAEFSKSNSSFHLRKASVHWSDSAVYFCVLSEDSNYQLIWGSGTKLIIKPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 2 - protein, 262 aa, Mus musculus
MFLWLQLDGMSQGEQVEQLPSILRVQEGSSASINCSYEDSASNYFPWYKQEPGENPKLIIDIRSNMERKQIQELIVLLDKKAKRFSLHITDTQPGDSAMYFCAAIFGGSNAKLTFGKGTKLSVKSNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 3 - protein, 131 aa, Mus musculus
MSCRLLLYVSLCLVETALMNTKITQSPRYLILGRANKSLECEQHLGHNAMYWYKQSAEKPPELMFLYNLKQLIRNETVPSRFIPECPDSSKLLLHISAVDPEDSAVYFCASSPHRGTMLSSSSDQGHDSPS

SEQ ID NO: 4 - protein, 306 aa, Mus musculus
MATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLSGGGTEVFFGKGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKKKNS

SEQ ID NO: 5 - protein, 330 aa, Mus musculus
MATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLSGGGTEVFFGKGTRLTVVGLRLSYASHHSSLTSQCRSECGTSEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKKKNS

SEQ ID NO: 6 - DNA, 20 bp, Mus musculus
ctctccagcaaccttcctca

SEQ ID NO: 7 - DNA, 20 bp, Mus musculus
ggctccttttggcttgaaga

SEQ ID NO: 8 - DNA, 34 bp, Mus musculus
aggcgcgccttcaactggaccacagcctcagcgt

SEQ ID NO: 9 - DNA, 34 bp, Mus musculus
aggcgcgccttcaggaattytttytyttgaccat

SEQ ID NO: 10 - DNA, 19 bp, Mus musculus
ggatctcatagaggatggt

SEQ ID NO: 11 - DNA, 31 bp, Mus musculus
aggcgcgcctggccacttgtcctcctctgaa

SEQ ID NO: 12 - DNA, 25 bp, Mus musculus
agcatgccatggtcctggcgctcct

SEQ ID NO: 13 - DNA, 27 bp, Mus musculus
agcatgccatggctacaaggctcctct

SEQ ID NO: 14 - DNA, 25 bp, Mus musculus
agcatgccatggttctatggctgca

(19) United States
(12) Patent Application Publication
(10) Pub. No.: US 2004/0171111 A1
(43) Pub. Date: Sep. 2, 2004

(54) Polypeptide of a p53 protein-specific murin a/t-cell receptor, nucleic acids coding therefor and use thereof

(76) Inventors: Thomas Stanislawski, Jugenheim (DE); Frank Schmitz, Bitburg (DE); Holger Voss, Ingelheim (DE); Matthias Theobald, Mainz-Kastel (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/469,552

(22) PCT Filed: Feb. 28, 2002
(86) PCT No.: PCT/EP02/02186

(30) Foreign Application Priority Data
Mar. 1, 2001 (DE) 101 09 855.3

(51) Int. Cl. C12P 21/02; C07K 14/705

(52) U.S. Cl. 435/69.1; 435/320.1; 435/325; 530/350; 536/23.5

(57) ABSTRACT

The invention relates to polypeptides of a murine α/β T-cell receptor mediating a p53 protein-specific T-cell response or functional variants or parts thereof or nucleic acids coding therefor, functional variants or parts thereof. Said polypeptides enable p53 protein-expressing cells to be recognized by T-cells provided with these genes, cytokines can be released, and T-cell induced lysis and/or apoptosis of tumor or leukemia cells can be performed.

### DESCRIPTION

The invention relates to polypeptides of the murine α/β T-cell receptor mediating a p53 protein-specific T-cell response, nucleic acids encoding these and their use in the therapy, diagnosis and/or prevention of diseases associated with the p53 protein.

Antigen recognition by T lymphocytes (CTL) is crucial for the generation and regulation of an effective immune response. The characteristic T-cell line marker is the T-cell antigen receptor (TCR). There are two defined types of TCR: One is a heterodimer of two disulfide-bonded polypeptides (α and β); the other is admittedly structurally similar, but consists of γ- and δ-polypeptides. Both receptors are associated with a set of five polypeptides, the CD3 complex, and thus together form the TCR complex (TCR-CD3 complex). The α/β-TCR is the most important functionally, since it is expressed in over 95% of all T-cells.

α/β T-cells can be subdivided into two different overlapping populations: A subgroup which carries the CD4 marker and mainly assists the immune response (TH) and a subgroup which carries the CD8 marker and is essentially cytotoxic (Tc). CD8+T-cells recognize antigens in association with MHC class I molecules. Such antigens can, inter alia, be tumor-specific or tumor-associated peptide antigens. After recognition of the peptide antigens, the cell concerned is destroyed by the T-cell lyzing the target cell and/or inducing apoptosis of these target cells or releasing cytokines (e.g. IL-2, IFN-γ).

Among the tumor-associated peptide antigens (TAA), which in the context of MHC class I molecules are presented on the surface of tumor cells, the “universal” TAA are of particular interest. These TAA are derived mainly from cellular proteins, which are weakly expressed in normal cells and overexpressed in tumor cells. These proteins include, inter alia, the “p53” protein whose expression is increased in approximately 50% of all human malignant disorders, in particular in a number of solid tumors, and whose turnover in accordance with proteasome-mediated degradation and subsequent MHC class I-associated presentation is increased.

Oligopeptides of the p53 protein can be presented to the cell-surface in context with MHC class I molecules and represent attractive target structures for CD8-positive T-cells.

One approach of developing immunotherapeutic procedures for the treatment of malignant oncoses is the identification of protein-specific TCRs. Such TCRs may, under certain conditions, provide T-cells with antigen specificity in general and tumor reactivity in particular, with the aim that said T-cells bring about the remission and the eradication of a certain tumor.

Weijtens et al. (“A retroviral vector system, ‘STITCH’; in combination with an optimized single chain antibody chimeric receptor gene structure allows efficient gene transduction and expression in human T-lymphocytes”, 1998, Gene Therapy, 5: 1995-1203) describe a retroviral vector system for the transduction of genes into activated T-lymphocytes. This system is used in order to bring about the expression of antibody-based chimeric receptors in the membrane of T-cells. These T-cells can then be employed against renal carcinoma cells, for example. Protein expression and thus successful vector transfer is determined by means of FACS analysis, while cytotoxicity assay give evidence of successful expression and function of the chimeric receptor. Similarly, Eshhar et al. (“Specific activation and targeting of cytotoxic-lymphocytes through chimeric single chains consisting of antibody-binding domains and the γ or ζ subunits of the immunoglobulin and T-cell receptors”, 1993, Proc. Natl. Acad. Sci. USA, 90: pp. 720-724) describe the preparation of tumor-specific lymphocytes and their use in immunotherapy on the basis of chimeras, which comprise the variable regions of an antibody with the constant region of TCR. It was possible for these chimeric genes to be expressed on the surface of cytolytic T-cell hybridomas and they brought about the secretion of interleukin-2 after contact with the antigen.

Clay et al. (“Efficient transfer of a tumor antigen-reactive TCR to human peripheral blood lymphocytes confers anti-tumor reactivity”; 1999, J. Immunology, 163: 507-513) describe the isolation of genes of the α-TCR and β-TCR chains of a MART-1 (25-35) specific TCR and its expression in human peripheral blood lymphocytes (PBLs). The lysis of various melanoma cell lines is described.

Darcy et al. (“Redirected perforin-dependent lysis of colon carcinoma by ex vivo genetically engineered CTL”, 2000, J. Immunol., 164, 3705-3712) describe an immuno-therapeutic procedure for colon carcinoma using scFv anti-CEA receptor transduced CTL, perforin and γ-IFN. The chimeric specific receptor construct is transduced into primary mouse T-lymphocytes by means of a retroviral vector. These cells were injected into mice (what is known as adoptive T-cell transfer) which had previously been inoculated with colon carcinoma cell lines.

From Theobald et al. (“Targeting p53 as a general tumor antigen”, 1995, Proc. Natl. Acad. Sci. USA 92: 11993-11997), the preparation of p53-specific cytotoxic T-cells after the injection of peptides from the wildtype sequence of p53 is known. By means of these T-cell lines, it was subsequently possible to lyze a selection of human tumor cells. The isolation of genes of specific TCRs of the lytic T-cells directed against p53 is not described. WO 97/32603 generally describes a process for the preparation of recombinant T lymphocytes, which express specific TCRs directed against tumor tissue. In this process, an HLA-transgenic mouse (in this case HLA-A2.1) is immunized with tumor-associated antigen in order thus to bring about the production of cytotoxic T lymphocytes which express specific TCRs on their surface. As tumor-associated antigens, peptides of various genes, such as Her-2/neu, Ras, p53, tyrosinase, MART, gp100, MAGE, BAGE and MUC-1 are described. From the Her-2/neu specific T lymphocytes, the nucleotide sequence which contains at least one variable region of the α- and β-chain of the corresponding nonhuman TCR is isolated and used in various genetic (inter alia “humanized”) TCR constructs. Thus, WO 97/32603 describes fusion proteins of variable regions of TCR with the ζ-region of CD3, CD8 or CD16, and also the use of flexible linkers of the amino acid sequence (GGGGS)₃.

A TCR directed specifically against the oligopeptide of amino acids 264-272 of p53 and its use is, however, neither mentioned nor suggested by the above-mentioned publications and in the other literature.

The present invention is therefore based on the object of making available the murine genes of the chains (α-TCR and β-TCR) of a novel TCR which is effectively directed against the p53 protein. These cause p53 protein-expressing cells to be recognized by T-cells which have been provided with said genes, cytokines to be secreted, and a T-cell-induced lysis and/or apoptosis of tumor or leukemia cells to be produced.

According to the invention, this object is achieved by the polypeptide of the murine α/β TCR mediating a p53 protein-specific T-cell response according to SEQ ID No. 1 to SEQ ID No. 5 or functional variants or parts thereof or nucleic acids encoding this, functional variants or parts thereof, in particular substitutions, deletions, insertions, additions, inversions and/or chemical or physical modifications of one or more amino acids or nucleic acids encoding these.

From a single-A2.1 transgenic mouse (([A2.1×C57BL/6]×C57BL/6)_{Fl}), the genes of the murine α or β TCR chains mediating a p53 protein-specific T-cell response were isolated, which mediate a specific T-cell response which is HLA-A2.1-restricted and directed against the peptide of the amino acids 264-272 of the p53 protein. The polypeptides of this TCR were hitherto unknown. The genes were inserted retrovirally into human peripheral blood lymphocytes (PBLs) as wild-type (WT) and the HLA-restricted antigen recognition was checked functionally by means of TCR-mediated cytotoxic lysis of various cell lines in the ⁵¹chromium-release test. It was possible to isolate, in addition to the “regular” P-TCR chain of the present invention, a P-TCR chain variant produced due to alternative mRNA maturation (described in Behlke and Loh “Alternative splicing of murine T-cell receptor β-chain transcripts”, 1986, Nature 322: 379-382). The T-cell population obtained was capable of recognizing peptide in nanomolar concentrations and efficiently destroying p53 transfectants and a variety of A2.1-positive malignant cells. The genes of the p53-specific TCR according to the invention are not included by the previously known suitable materials for the diagnosis—such as, for example, the indication—and/or the treatment—such as, for example, the modulation—of diseases connected with p53 protein or for the identification of pharmacologically active substances, such that completely novel therapeutic approaches result from this invention.

Liu et al. (“Targeting of human p53-overexpressing tumor cells by an HLA A*0201-restricted murine T-cell receptor expressed in Jurcat T Cells”, 2000, Cancer Res. 60, 693-701) describe the preparation of a p53-specific TCR, based on injected peptides. The T-cells obtained are described as being suitable for immunotherapy of cancer. However, the TCR described by Liu et al. is directed against a peptide of amino acids 149-157 of p53. Since the TAA epitope recognized by the TCRs is of crucial importance for the therapeutic action and side effects, the presence of further effective and, up to now, unknown TCRs directed against other peptides/epitopes of D53 is all the more surprising.

A further aspect of the invention relates to a fusion protein, comprising the polypeptide according to the invention or functional variants or parts thereof or nucleic acids encoding this, functional variants or parts thereof.

The fusion protein can be characterized in that it comprises the ζ-region von CD3 oder CD8 or CD16 or parts thereof, in particular the ζ-region of human CD3 or CD8 or CD16 or parts thereof. A fusion protein according to the invention is preferred which comprises a flexible linker (Whitlow et al., “An improved linker for single-chain Fv with reduced aggregation and enhanced proteolytic stability”, Prot. Engin. 6(8), pp. 989-995, 1993), in particular a linker of the amino acid sequence (GGGGS)₃. In particular, the fusion protein according to the invention can comprise the ζ-chain of the CD3-complex or ITAM motif of the ζ-chain or parts thereof, in particular the ζ-chain of human CD3 or parts thereof. The fusion protein can furthermore be characterized in that it comprises CD8α or the Lck-binding motif of CD8α or parts thereof, in particular of human CD8α.

The fusion protein according to the invention can furthermore be a chimeric partially or completely humanized α and/or β TCR chain. A further aspect of the invention relates to a fusion protein which is a single-chain TCR. A fusion protein according to the invention is preferred which comprises a flexible linker, in particular a linker of the amino acid sequence (GGGGS)₃. The fusion protein according to the invention can, however, also be characterized in that it is an α/β-TCR.

A further subject of the invention is a process for the preparation of a fusion protein for the diagnosis and/or treatment of diseases connected with p53 protein or for the identification of pharmacologically active substances, e.g. in a suitable host cell, in which a nucleic acid according to the invention is used.

Fusion proteins are prepared here which contain the polypeptides according to the invention described above, where the fusion proteins themselves already have the function of a polypeptide of the invention or the specific function is functionally active only after removal of the fusion portion. These especially include fusion proteins having a proportion of about 1-200, preferably about 1-150, in particular about 1-100, especially about 1-50 foreign amino acids. Examples of such peptide sequences are prokaryotic peptide sequences, which can be derived, for example, from the galactosidase of E. coli. Furthermore, viral peptide sequences, such as, for example, of the bacteriophage M13, can also be used in order thus to produce fusion proteins for the “phage display” process known to the person skilled in the art.

For the purification of the proteins according to the invention, a further polypeptide (“tag”) can be added. Protein tags according to the invention allow, for example, high-affinity absorption on a matrix, stringent washing with suitable buffers without eluting the complex to a noticeable extent, and subsequently the elution of the absorbed complex in a controlled manner. Examples of the protein tags known to the person skilled in the art are a (His)₆ tag, a Myc tag, a FLAG tag, a Strep tag, a Strep tag II, a hemagglutinin tag, glutathione transferase (GST) tag, intein with an affinity chitin-binding tag or maltose-binding protein (MBP) tag. These protein tags can be located N—, C-terminally and/or internally.

Beside the natural polypeptides isolated from cells, all polypeptides according to the invention or their parts can have been prepared under cell-free conditions, e.g. by synthesis or by in vitro translation. Thus, the entire polypeptide or parts thereof can be synthesized, for example, with the aid of the classical synthesis (Merrifield technique). Parts of the polypeptides according to the invention are suitable, in particular, for the obtainment of antisera, with the aid of which suitable gene expression banks can be searched in order thus to obtain further functional variants of the polypeptide according to the invention.

The invention also relates to polypeptides which are derivatives of an antibody having a specificity for the p53 peptide antigen (AA 264-272), preferably presented in the context of HLA-A2.1.

The invention furthermore comprises retro-inverse peptides or pseudopeptides according to the polypeptide sequence of SEQ ID No. 1 to SEQ ID No. 5 or functional variants or parts thereof. Instead of the —CO—NH— peptide bonds, these peptides have —NH—CO— bonds.

The object of the invention is furthermore achieved by a nucleic acid according to the invention, which is a DNA, RNA, PNA (peptide nucleic acid) or p-NA (pyranosyl nucleic acid), preferably a DNA, in particular is a double-stranded DNA having a length of at least 8 nucleotides, preferably having at least 12 nucleotides, in particular having at least 24 nucleotides. The nucleic acid can be characterized in that the sequence of the nucleic acid has at least one intron and/or one polyA sequence. It can also be present in the form of its antisense sequence.

For the expression of the gene concerned, in general a double-stranded DNA is preferred, the DNA region coding for the polypeptide being particularly preferred. This region begins with the first start codon (ATG) In a Kozak consensus sequence (Kozak 1987, Nucleic Acids Res. 15:8125-48) up to the next stop codon (TAG, TGA bzw. TAA), which is in the same reading frame with respect to the ATG. A further use of the nucleic acid sequences according to the invention is the construction of anti-sense oligonucleotides (Zheng and Kemeny 1995, Clin. Exp. Immunol. 100:380-382; Nellen and Lichtenstein 1993, Trends Biochem. Sci. 18:419-23) and/or ribozymes (Amarzguioui, et al. 1998, Cell. Mol. Life Sci. 54:1175-202; Vaish, et al., 1998, Nucleic Acids Res. 26:5237-42; Persidis, 1997, Nat. Biotechnol. 15:921-2; Couture and Stinchcomb 1996, Trends Genet. 12:510-5). Using “anti-sense” oligonucleotides, it is possible to decrease the stability of the nucleic acid according to the invention and/or to inhibit the translation of the nucleic acid according to the invention. Thus it is possible, for example, to decrease the expression of the corresponding genes in cells both in vivo and in vitro. Oligonucleotides can therefore be suitable as a therapeutic. This strategy is suitable, for example, also for the skin, epidermal and dermal cells, in particular if the “antisense” oligonucleotides are complexed with liposomes (Smyth et al. 1997, J. Invest. Dermatol. 108:523-6; White et al. 1999, J. Invest. Dermatol. 112:699-705; White et al. 1999, J. Invest. Dermatol. 112:887-92). For use as a probe or as an “antisense” oligonucleotide, a single-stranded DNA or RNA is preferred.

Beside the natural nucleic acids isolated from cells, all nucleic acids according to the invention or their parts can also have been prepared synthetically. Furthermore, for carrying out the invention, a nucleic acid can be used which has been prepared synthetically. Thus, the nucleic acid according to the invention can be synthesized, for example, chemically with the aid of the protein sequences described in SEQ ID No. 1 to SEQ ID No. 5 making use of the genetic code, e.g. according to the phosphotriester method (see e.g. Uhlmann & Peyman 1990, Chemical Reviews 90:543-584).

Oligonucleotides are as a rule degraded rapidly by endo- or exonucleases, in particular by DNases and RNases occurring in the cell. It is therefore advantageous to modify the nucleic acid in order to stabilize it against degradation, so that a high concentration of the nucleic acid is maintained in the cell over a long period of time (Beigelman et al. 1995, Nucleic Acids Res. 23:3989-94; Dudycz 1995, WO 95/11910; Macadam et al. 1998, WO 98/37240; Reese et al. 1997, WO 97/29116). Typically, such a stabilization can be obtained by the introduction of one or more inter-nucleotide phosphate groups or by the introduction of one or more non phosphorus internucleotides.

Suitable modified internucleotides are summarized in Uhlmann and Peymann (1990, Chem. Rev. 90:544) (see also Beigelman et al. 1995, Nucleic Acids Res. 23:3989-94; Dudycz 1995, WO 95/11910; Macadam et al. 1998, WO 98/37240; Reese et al. 1997, WO 97/29116). Modified internucleotide phosphate radicals and/or non phosphorus ester bonds in a nucleic acid which can be employed in one of the uses according to the invention, contain, for example, methylphosphonate, phosphorothioate, phosphor-amidate, phosphorodithioate, phosphate esters, while non phosphorus internucleotide analogs, for example, contain siloxane bridges, carbonate bridges, carboxy-methyl esters, acetamidate bridges and/or thio bridges. It is also intended that this modification improves the stability of a pharmaceutical composition which can be employed in one of the uses according to the invention.

A further aspect of the present invention relates to a vector, preferably in the form of a plasmid, shuttle vector, phagemid, cosmid, expression vector, adenoviral vector, retroviral vector (Miller, et al. “Improved retroviral vectors for gene transfer and expression”, BioTechniques Vo'l. 7, No. 9, p 980, 1989) and/or a vector having gene therapy activity, which contains a nucleic acid according to the invention.

Thus the nucleic acid according to the invention can be contained in a vector, preferably in an expression vector or vector active in gene therapy. Preferably, the vector active in gene therapy contains T-cell-specific regulatory sequences, which are bound functionally to the nucleic acid according to the invention. The expression vectors can be prokaryotic or eukaryotic expression vectors. Examples of prokaryotic expression vectors are, for expression in E. coli, for example, the vectors pGEM or pUC derivatives and for eukaryotic expression vectors for expression in Saccharomyces cerevisiae, for example, the vectors p426Met25 or p426GAL1 (Mumberg et al. 1994 Nucleic Acids Res. 22:5767-5768), for expression in insect cells, for example, Baculovirus vectors such as disclosed in EP-B1-0 127 839 or EP-B1-0 549 721, and for expression in mammalian cells, for example, the vectors Rc/CMV and Rc/RSV or SV40 vectors, which are all generally obtainable.

In general, the expression vectors also contain promoters suitable for the respective host cell, such as, for example, the trp promoter for expression in E. coli (see, for example, EP-B1-0 154 133), the Met 25, GAL 1 or ADH2 promoter for expression in yeasts (Russel et al. 1983, J. Biol. Chem. 258:2674-2682; Mumberg, supra), the Baculovirus polyhedrin promoter for expression in insect cells (see e.g. 13. EP-B1-0 127 839). For expression in mammalian cells, suitable promoters are, for example, those which allow a constitutive, regulatable, tissue-specific, cell cycle-specific or metabolically specific expression in eukaryotic cells. Regulatable elements according to the present invention are promoters, activator sequences, enhancer, silencer and/or repressor sequences. Examples of suitable regulatable elements which make possible constitutive expression in eukaryotes are promoters which are recognized by the RNA polymerase III or viral promoters, CMV enhancer, CMV promoter, SV40 promoter or LTR promoters, for example of MMTV (mouse mammary tumour virus; Lee et al. 1981, Nature, 214:228-232) and further viral promoter and activator sequences, derived from, for example, HBV, HCV, HSV, HPV, EBV, HTLV or HIV. Examples of regulatable elements which make possible regulatable expression in the eukaryotes are the tetracycline operator in combination with an appropriate repressor (Gossen et al. 1994 Curr. Opin. Biotechnol. 5:516-20).

Examples of regulatable elements which make possible T-cell-specific expression in eukaryotes are promoters or activator sequences of promoters or enhancers of those genes which code for proteins which are only expressed in these cell types.

Examples of regulatable elements which make possible cell cycle-specific expression in eukaryotes are promoters of the following genes: cdc25, cyclin A, cyclin E, cdc2, E2F, B-myb or DHFR (Zwicker and Müller 1997, Trends Genet. 13:3-6). Examples of regulatable elements which make possible metabolically specific expression in eukaryotes are promoters which are regulated by hypoxia, by glucose deficiency, by phosphate concentration or by heat shock. The vector according to the invention can be used for the transfection of a host cell which is preferentially a T-cell. Particularly preferred is a host cell which is characterized in that it expresses a polypeptide or fusion protein according to the invention on its surface.

In order to make possible the introduction of the nucleic acids according to the invention and thus the expression of the polypeptide in a eukaryotic or prokaryotic cell by transfection, transformation or infection, the nucleic acid can be present as a plasmid, as part of a viral or nonviral vector. Suitable viral vectors in this case are particularly: retroviruses baculoviruses, vaccinia viruses, adenoviruses, adeno-associated viruses and herpesviruses. Suitable nonviral carriers in this case are particularly: virosomes, liposomes, cationic lipids, or poly-lysine-conjugated DNA.

Examples of vectors active in gene therapy are virus vectors, for example adenovirus vectors or retroviral vectors (Lindemann et al., 1997, Mol. Med. 3:466-76; Springer et al. 1998, Mol. Cell. 2:549-58).

A preferred mechanism for expressing polypeptides according to the invention in vivo is viral gene transfer, in particular with the aid of retroviral particles. These are preferably utilized to provide appropriate target cells, preferably T-lymphocytes, of the patient ex vivo with the genes or nucleotide sequences coding for polypeptides according to the invention by transduction. The target cells can afterwards be reinfused into the patient again in the sense of an adoptive cell transfer in order to take over tumoricidal and/or immunomodulating effector functions using the de novo inserted specificity. Recently, in this way very good gene therapy results were achieved in the treatment of the SCID-X1 disease characterized by immunoincompetence in newborn children in whom hematological precursor cells were provided retrovirally with an analogous intact transgene of a nonfunctional mutated variant of the γ-chain gene occurring in the children, which is essential for the differentiation into the various effector cells of the adaptive immune system (Cavazzana-Calvo et al., 2000).

There is furthermore the possibility of carrying out the gene transfer in vivo, on the one hand by preferentially stereotactic injection of the infectious particles, on the other hand by direct administration of virus-producing cells (Oldfield, et al. Hum. Gen. Ther., 1993, 4:39-69).

The viral vectors often employed for the transfer of genes are according to the present state of the art mainly retroviral, lentiviral, adenoviral and adeno-associated viral vectors. These are cyclic nucleotide sequences derived from natural viruses, in which at least the viral structural protein-coding genes are replaced by the construct to be transferred.

Retroviral vector systems create the prerequisite for a long-lasting expression of the transgene by the stable, but undirected integration into the host genome. Vectors of the younger generation possess no irrelevant and potentially immunogenic proteins, in addition there is no preexisting immunity of the recipient to the vector. Retroviruses contain an RNA genome which is packaged in a lipid coat which consists of parts of the host cell membrane and of virus proteins. For the expression of viral genes, the RNA genome is reverse-transcribed and integrated into the target cell DNA with the enzyme integrase. This can afterwards be transcribed and translated by the infected cell, whereby viral constituents result which combine to give retroviruses. RNA is exclusively then inserted into the newly formed viruses. The genome of the retroviruses possesses three essential genes: gag, which codes for viral structural proteins, “group-specific antigens”, pol for enzymes such as reverse transcriptase and integrase and env for the coat protein (“envelope”), which is responsible for the binding of the host-specific receptor. The production of the replication-incompetent viruses takes place after transfection in “packaging cell lines”, which have additionally been equipped with the gag/pol-coding genes and express these “in trans” and thus complement the formation of replication-incompetent (i.e. gag/pol-deleted) transgenic virus particles. An alternative is the co-transfection of the essential virus genes, only the vector containing the transgene carrying the packaging signal.

The separation of these genes on the one hand makes possible the arbitrary combination of the gal/pol-reading frame with env reading frames obtained from various strains, whereby pseudotypes having modified host tropism result, on the other hand the formation of replication-competent viruses within packaging cells can be drastically reduced thereby. The coat protein derived from “gibbon ape leukemia virus” (GALV), which is used in the present case, is able to transduce human cells and is established in the packaging cell line PG13 with an amphotrophic host range (Miller et al., 1991). Additionally, the safety is increased by selective deletion of nonessential virus sequences for the prevention of a homologous recombination and thus the production of replication-competent particles.

Novel, nonviral vectors consist of autonomous, self-integrating DNA sequences, the transposons, which are inserted into the host cell by, for example, liposomal transfection and have for the first time been employed successfully for the expression of human transgenes in mammalian cells (Yant et al., 2000).

Vectors active in gene therapy can also be obtained by complexing the nucleic acid according to the invention with liposomes, since thereby a very high transfection efficiency, in particular of skin cells, can be achieved (Alexander and Akhurst, 1995, Hum. Mol. Genet. 4:2279-85). Lipofection comprises preparing small unilamellar vesicles from cationic lipids by ultrasound treatment of the liposome suspension. The DNA is bound Tonically to the liposome surface and in such a ratio that a positive net charge remains and the plasmid DNA is 100% complexed by the liposomes. In addition to the lipid mixtures DOTMA (1,2-dioleyloxpropyl-3-trimethylammonium bromide) and DPOE (dioleoylphosphatidylethanolamine) used by Felgner et al. (1987, supra), numerous new lipid formulations have been synthesized and assayed for their efficiency of transfecting various cell lines since then (Behr et al. 1989, Proc. Natl. Acad. Sci. USA 86:6982-6986; Felgner et al. 1994, J. Biol. Chem. 269:2550-25561; Gao and Huang. 1991, Biochim. Biophys. Acta 1189:195-203). Examples of the new lipid formulations are DOTAP N-[1-(2,3-dioleoyloxy)propyl]-N,N,N-trimethylammoniumethyl sulfate or DOGS (TRASFECTAM; dioctadecyl-amidoglycylspermine). Excipients which increase the transfer of nucleic acids to the cells can, for example, be proteins-or peptides which are bound to DNA or synthetic peptide-DNA molecules which make possible the transport of the nucleic acid into the nucleus of the cell (Schwartz et al. 1999, Gene Therapy 6:282; Brandén et al. 1999, Nature Biotech. 17:784). Excipients also include molecules which make possible the release of nucleic acids into the cytoplasm of the cell (Kiehler et al. 1997, Bioconj. Chem. 8:213) or, for example, liposomes (Uhlmann and Peymann 1990, supra). Another particularly suitable form of gene therapy vectors can be obtained by applying the nucleic acid according to the invention to gold particles and shooting these into tissue, preferably into the skin, or cells with the aid of the “gene gun” (Wang et al., 1999, J. Invest. Dermatol., 112:775-81).

For the gene therapy application of the nucleic acid according to the invention, it is also advantageous if the part of the nucleic acid which codes for the polypeptides contains one or more noncoding sequences including intron sequences, preferably between the promoter and start codon of the polypeptide, and/or a polyA sequence, in particular the naturally occurring polyA sequence or an SV40 virus polyA sequence, especially at the 3′-end of the gene, since by this means a stabilization of the mRNA can be achieved (Jackson 1993, Cell 74:9-14 and Palmiter et al. 1991, Proc. Natl. Acad. Sci. USA 88:478-482).

A further subject of the present invention is a host cell, in particular a T-cell, which is transformed using a vector according to the invention or another gene construct according to the invention. Host cells can be either prokaryotic or eukaryotic cells, examples of prokaryotic host cells are E. coli and of eukaryotic cells are Saccharomyces cerevisiae or insect cells.

A particularly preferred transformed host cell is a transgenic T-precursor cell or a stem cell, which is characterized in that it comprises a gene construct according to the invention or an expression cassette according to the invention. Processes for the transformation of host cells and/or stem cells are well known to the person skilled in the art and include, for example, electroporation, microinjection or transduction. A particularly preferred transformed host cell is a patient's own T-cell, which after removal is transfected or transduced using a gene construct according to the invention. Host cells according to the invention can in particular be obtained by removing from the patient one or more cells, preferentially T-cells, in particular CD8⁺-T-cells, which are then transfected or transduced ex vivo using one or more genetic constructs according to the invention in order thus to obtain host cells according to the invention. The specific T-cells generated ex vivo can then subsequently be reimplanted into the patient. The process is thus similar to the process described in Darcy et al. (“Redirected perforin-dependent lysis of colon carcinoma by ex vivo genetically engineered CTL”, J. Immunol. 2000, 164:3705-3712) using CTL transduced by scFv anti-CEA receptor, perforin and γ-IFN.

A further preferred process according to the invention for the identification of p53 protein-specific antigens is characterized in that p53-presenting tumor cells or fractions thereof are combined with a host cell according to the invention under conditions in which the tumor cells or fractions thereof are only lyzed if the tumor presents p53 protein-specific antigen for which the expressed polypeptide or fusion protein is specific.

A further aspect of the invention relates to a process for the preparation of an antibody, preferably of a polyclonal or monoclonal antibody, for the diagnosis and/or treatment of diseases associated with p53 protein or for the identification of pharmacologically active substances, characterized in that an antibody-producing organism is immunized with a polypeptide according to the invention or functional equivalents thereof or parts thereof having at least 6 amino acids, preferably having at least 8 amino acids, in particular having at least 12 amino acids, or with a nucleic acid according to the invention.

The process is carried out according to methods generally known to the person skilled in the art by immunizing a mammal, for example a rabbit, with the polypeptide according to the invention or the parts thereof mentioned or nucleic acid(s) encoding this (these), if appropriate in the presence of, for example, incomplete Freund's adjuvant and/or aluminum hydroxide gels (see, for example, Diamond et al. 1981, The New England Journal of Medicine, pp. 1344-1349). The polyclonal antibodies formed in the animal on account of an immunological reaction can then be easily isolated from the blood according to generally known methods and purified, for example, by means of column chromatography. Monoclonal antibodies can be prepared, for example, according to the known method of Winter & Milstein (1991, Nature 349:293-299).

A further subject of the present invention is an antibody for the diagnosis, prognosis and therapy-optimization of diseases associated with p53 protein or for the identification of pharmacologically active substances, which is directed against a polypeptide according to the invention and specifically reacts with the polypeptides according to the invention, where the abovementioned parts of the polypeptide are either themselves immunogenic or can be made immunogenic or increased in their immunogenicity by coupling to suitable carriers, such as, for example, bovine serum albumin. This antibody is either polyclonal or monoclonal, a monoclonal antibody is preferred. The term antibody is understood according to the present invention as also meaning prepared by genetic engineering and optionally modified antibodies or antigen-binding parts thereof, such as, for example, chimeric antibodies, humanized antibodies, multi-functional antibodies, bi- or oligospecific antibodies, single-stranded antibodies, F(ab) or F(ab)₂ fragments (see, for example, EP-B1-0 368 684, U.S. Pat. No. 4,816,567, U.S. Pat. No. 4,816,397, WO 88/01649, WO 93/06213, WO 98/24884). The antibodies according to the invention can be used for the diagnosis, therapy monitoring and/or treatment of diseases associated with p53 protein or for the identification of pharmacologically active substances.

The present invention furthermore relates to a process for the production of a medicament for the treatment of diseases associated with p53 protein, characterized in that at least one nucleic acid, at least one polypeptide, at least one host cell or at least one antibody as claimed in one of the aforementioned claims is combined, together with suitable additives and excipients.

The present invention furthermore relates to a medicament prepared according to this process for the treatment of diseases associated with p53 protein, which contains at least one nucleic acid, at least one polypeptide or at least one antibody according to the present invention, if appropriate together with suitable additives and excipients. The invention furthermore relates to the use of this medicament for the treatment of diseases associated with p53 protein.

The therapy of diseases associated with p53 protein can be carried out in a conventional manner, e.g. by means of infusions or injections which contain the medicaments according to the invention. The administration of the medicaments according to the invention can furthermore be optionally carried out in the form of liposome complexes or gold particle complexes. The treatment by means of the medicaments according to the invention can, however, also be carried out via oral dosage forms, for example tablets or capsules, via the mucous membranes, for example the nose or the oral cavity, or in the form of dispositories implanted under the skin. Transdermal therapeutic systems are known, for example, from EP 0 944 398 A1, EP 0 916 336 A1, EP 0 889 723 A1 or EP 0 852 493 A1. The (poly)peptides and their derivatives according to the invention can also be employed for making patients with diseases, in particular oncoses, which are connected with p53 immunocompetent in a targeted manner, in order to achieve the induction of, production of and expansion of p53.264-272-specific cytotoxic T-lymphocytes and specifically to destroy the tumor and leukemia cells of the oatients concerned. Such diseases include, for example, solid oncoses, lymphohemato-poietic neoplasias, malignant hemato-logical diseases or myeloblastic crises.

In a particularly preferred type of treatment, one or more cells, preferentially T-cells, in particular CD8⁺-T-cells are taken from the patient, which are then transfected or transduced ex vivo with one or more genetic constructs according to the invention. The specific T-cells generated ex vivo can than subsequently be reinfused or transplanted into the patient. The process is thus similar to the immunotherapeutic process described in Darcy et al. (“Redirected perforin-dependent lysis of colon carcinoma by ex vivo genetically engineered CTL”, 2000, J. Immunol., 164:3705-3712) in the case of colon carcinomas using CTL transduced by scFv anti-CEA receptor, perforin and γ-IFN.

A further aspect of the invention relates to a process for the production of a test for the discovery of functional interactors in connection with p53 protein-associated diseases, which is characterized in that at least one nucleic acid, at least one polypeptide or at least one antibody according to the present invention is combined, together with suitable additives and excipients.

The term “functional interactors” within the meaning of the present invention is to be understood as meaning all those molecules, compounds and/or compositions and substance mixtures which can interact with the nucleic acids, polypeptides or antibodies according to the invention, if appropriate together with suitable additives and excipients, under suitable conditions. Possible interactors are simple chemical organic or inorganic molecules or compounds, but can also include peptides, proteins or complexes thereof. On account of their interaction, the functional interactors can influence the function(s) of the nucleic acids, polypeptides or antibodies in vivo or in vitro or alternatively only bind to the nucleic acids, polypeptides or antibodies according to the invention or enter into other interactions of a covalent or noncovalent manner with them.

The invention furthermore comprises a test produced according to the invention for the identification of functional interactors in connection with diseases associated with p53 protein, which contains at least one nucleic acid, at least one polypeptide or at least one antibody according to the present invention, if appropriate together with suitable additives and excipients. Often, the pathological behavior of the cells in vitro can thus be imitated and substances can be sought which restore the normal behavior of the cells and which possess a therapeutic potential. Moreover, this test system can be utilized for the screening of substances which inhibit an interaction between the polypeptide according to the invention and a functional interactor.

A subject of the present invention is also a medicament for the indication, such as, for example, diagnosis, and therapy of diseases associated with p53 protein, which contains a nucleic acid according to the invention or a polypeptide according to the invention and, if appropriate, suitable additives or excipients, and a process for the production of such a medicament for the treatment of diseases associated with p53 protein, in which a nucleic acid according to the invent-ion or a polypeptide according to the invention is formulated with a pharmaceutically acceptable carrier.

Possible therapeutics and/or prophylactics are in particular vaccines, recombinant particles or injections or infusion solutions, which as active compound (a) contain the TCR polypeptide according to the invention and/or its derivatives and/or (b) a nucleic acid according to the invention and/or (c) T-lymphocytes produced in vitro or ex vivo, which contain a TCR specifically directed against p53.264-272.

For gene therapy application in humans, a medicament and/or recombinant particle is especially suitable which contains the nucleic acid according to the invention in naked form or in the form of one of the vectors active in gene therapy described above or in a form complexed with liposomes or gold particles. The pharmaceutical carrier is, for example, a physiological buffer solution, preferably having a pH of about 6.0-8.0, preferably of about 6.8-7.8 in particular of about 7.4 and/or an osmolarity of about 200-400 milliosmol/liter, preferably of about 290-310 milliosmol/liter. Additionally, the pharmaceutical carrier can contain suitable stabilizers, such as, for example, nuclease inhibitors, preferably complexing agents such as EDTA and/or other excipients known to the person skilled in the art.

A further subject of the invention relates to a process for the preparation of a polypeptide for the diagnosis and/or treatment of diseases connected with p53 protein or for the identification of pharmacologically active substances in a suitable host cell, which is characterized in that a nucleic acid according to the invention is expressed in a suitable manner.

The polypeptide is thus prepared, for example, by expression of the nucleic acid according to the invention in a suitable expression system, as already described above, according to the methods generally known to the person skilled in the art. Suitable host cells are, for example, the E. coli strains DHS, HB101 or BL21, the yeast strain Saccharomyces cerevisiae, insect cell lines, e.g. from Spodoptera frugiperda, or the animal cells COS, Vero, 293, HaCaT, and HeLa, which are all generally obtainable.

A diagnostic according to the invention for therapy monitoring contains the polypeptide according to the invention or the immunologically active parts thereof described in greater detail above. The polypeptide or the parts thereof, which are preferably bound to a solid-phase, e.g. of nitrocellulose or Nylon, can be brought into contact in vitro with the body fluid to be investigated, e.g. blood, in order thus to be able to react, for example, with autoimmune antibodies or tumor and leukemia cells. The antibody-antigen complex can then be detected, for example, with the aid of labeled anti-human-IgG or anti-human-IgM antibodies. The label is, for example, an enzyme, for example peroxidase, which catalyzes a color reaction, or is another suitable label. The presence and the amount of autoimmune antibodies present can thus be detected easily and rapidly by means of the color reaction.

Another diagnostic for therapy monitoring contains the antibodies according to the invention themselves. With the aid of these antibodies, for example, a tissue sample can be easily and rapidly investigated as to whether the polypeptide concerned is present in an increased amount, in order thereby to diagnose diseases connected with p53 protein and to obtain indications for therapy. In this case, the antibodies according to the invention are labeled, for example, with an enzyme, as already described above. The specific antibody-antigen complex can thereby be easily and likewise rapidly detected by means of an enzymatic color reaction.

A further diagnostic according to the invention comprises a probe, preferably a DNA probe, and/or primers. This opens up a further possibility of obtaining the nucleic acids according to the invention, for example by isolation from a suitable gene bank with the aid of a suitable probe (see, for example, Sambrook et al. 1989, “Molecular Cloning. A Laboratory Manual” 2^{nd} edn., Cold Spring Harbor Laboratory, Cold Spring Harbor, N.Y. chapter 8 page 8.1 to 8.81, chapter 9 page 9.47 to 9.58 and chapter 10 page 10.1 to 10.67).

Suitable probes are, for example, DNA or RNA fragments having a length of about 100-1000 nucleotides, preferably having a length of about 200-500 nucleotides, in particular having a length of about 300-400 nucleotides, whose sequence can be derived from the polypeptides according to SEQ ID No. 1 to SEQ ID No. 5 of the sequence listing.

Alternatively, with the aid of the derived nucleic acid sequences oligonucleotides can be synthesized which are suitable as primers for a polymerase chain reaction. Suitable primers are, for example, DNA fragments having a length of about 10-100 nucleotides, preferably having a length of about 15 to 50 nucleotides, in particular having a length of 20-30 nucleotides, whose sequence can be derived from the polypeptides according to SEQ ID No. 1 to SEQ ID No. 5 of the sequence listing with the aid of the corresponding cDNA sequences according to the genetic code.

The term “coding nucleic acid” relates to a DNA sequence which codes for an isolable bioactive polypeptide according to the invention or a precursor. The polypeptide can be encoded by a sequence of full-length or any part of the coding sequence, as long as the specific, for example enzymatic, activity is retained.

It is known that small changes in the sequence of the nucleic acid according to the invention can be present, for example due to the degeneration of the genetic code, or that nontranslated sequences can be appended at the 5′ and/or 3′-end of the nucleic acid without its activity being significantly changed. This invention therefore also comprises “functional variants” of the nucleic acids according to the invention.

By the term “functional variants”, all DNA sequences are denoted which are complementary to a DNA sequence, which hybridize under stringent conditions with a derived reference sequence or parts thereof, in particular the hypervariable V(D)JC region, and have a similar or identical activity to the corresponding polypeptide according to the invention.

“Stringent hybridization conditions” are to be understood as meaning those conditions under which a hybridization takes place at 60° C. in 2.5×SSC buffer, followed by a number of washing steps at 37° C. in a lower buffer concentration, and remains stable.

The term “functional variants” within the meaning of the present invention is understood as meaning polypeptides which are related functionally to the polypeptides according to the invention, i.e. have structural features of the polypeptides. Examples of functional variants are the corresponding polypeptides which originate from organisms other than the mouse, that is the human, or, preferably from nonhuman mammals such as, for example, monkeys, pigs and rats, or else birds, for example chickens. Other examples of functional variants are polypeptides which are encoded by different alleles of the gene, in various individuals or in various organs of an organism. For the purposes of the present invention, functional variants are, in particular, polypeptides which recognize the identical epitope of the p53 protein as the TCR of the present invention. In a further sense, these are also understood as meaning polypeptides which have a sequence homology, in particular a sequence identity, of about 70%, preferably about 80%, in particular about 90%, especially about 95% to the polypeptide having the amino acid sequence according to one of SEQ ID No. 1 to SEQ ID No. 5 and/or to DNA sequences derived with the aid of the peptide sequences. Among these are also included deletions, inversions, additions, substitutions, insertions and chemical and/or physical modifications or parts of the polypeptide in the range from about 1-60, preferably from about 1-30, in particular from about 1-15, especially from about 1-5 amino acids. For example, the first amino acid methionine can lack without the function of the polypeptide being significantly modified.

The invention will now be illustrated further with the aid of the attached examples and figures, without being restricted by this. The figures show:

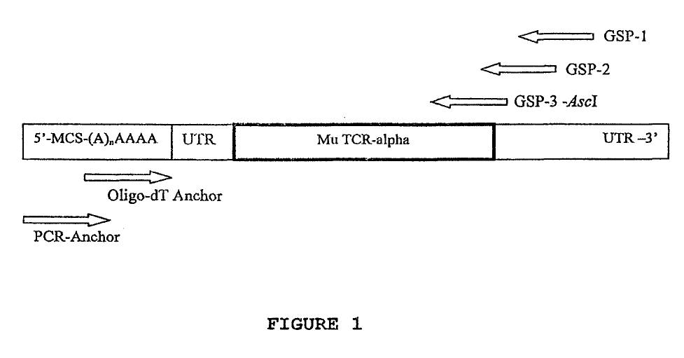

FIG. 1: Diagram of the primer positions for preparation of full-length TCR α-chain;

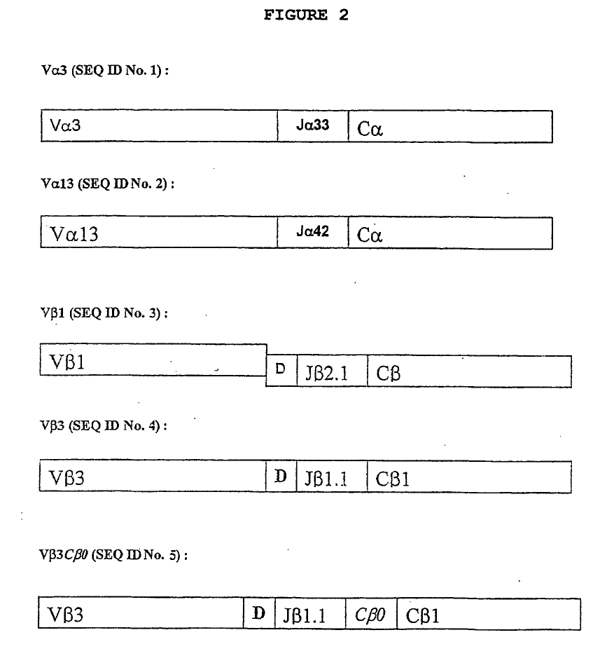

FIG. 2: Depiction of the TCR chains prepared. The nomenclature of the variable segments (V alpha/beta) was according to Arden et al. (Immunogenetics 1995, 42:501-530), that of the J segments and the constant domains was according to the ImMunoGeneTics Database (http://imgt.cines.fr:8104). Based on their sequence, TCR chains Vα3, Vα13, Vβ3 and Vβ3Cβ0 are productive, Vβ1 on the other hand, has a frame shift in the recombinant region V-D-J and is subsequently non productive for a TCR β-chain polypeptide. Cβ0 is the insertion produced by alternative splicing. We were unable to assign the constant domain of the Vβ1 chain to any subfamily, since only the truncated form was prepared and differentiation was therefore not possible.

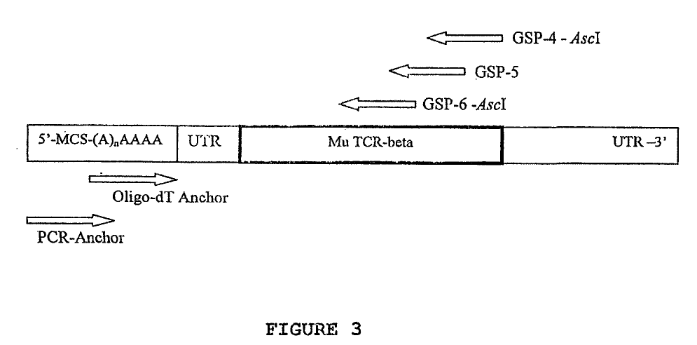

FIG. 3: Positions of the primers for preparation of the truncated TCR β-chains in the 5′-RACE PCR.

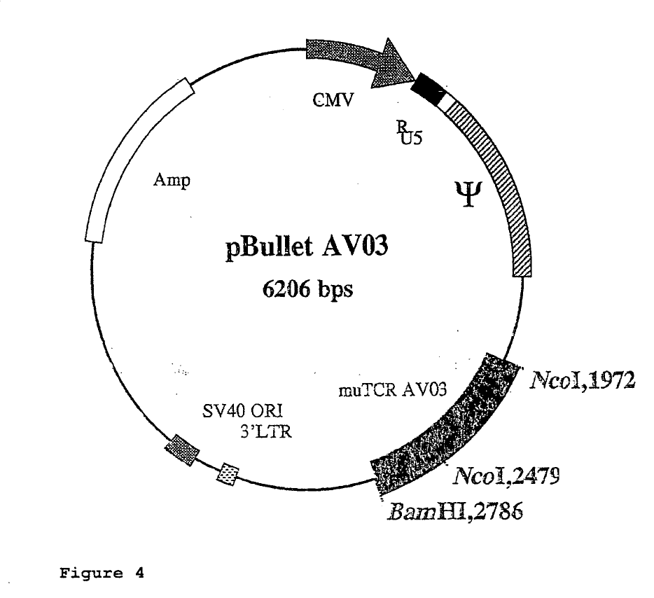

FIG. 4: Depiction of the viral vector pBullet AV03 for expressing wild-type (Wt) murine (mu) TCR Vα3 chain. The wild-type Vα3 chain was cloned, as described in the text, via restriction enzyme cleavage sites NcoI and BamHI into the retroviral vector pBullet.

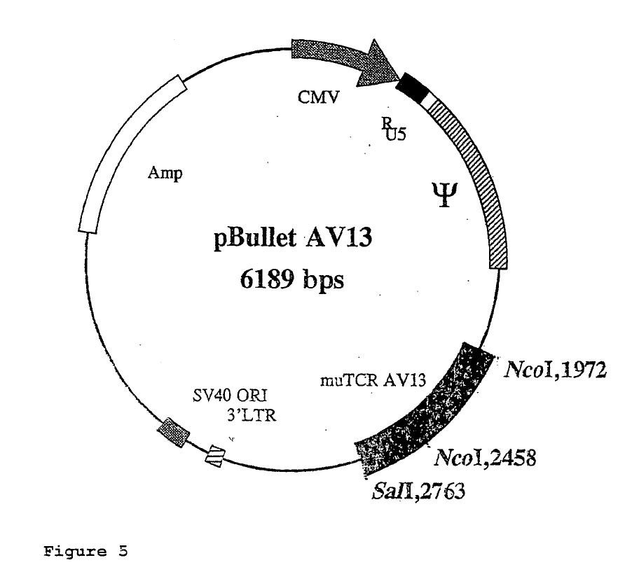

FIG. 5: Depiction of the Wt muTCR Vα13 chain which was cloned via restriction enzyme sites NcoI and SalI as described.

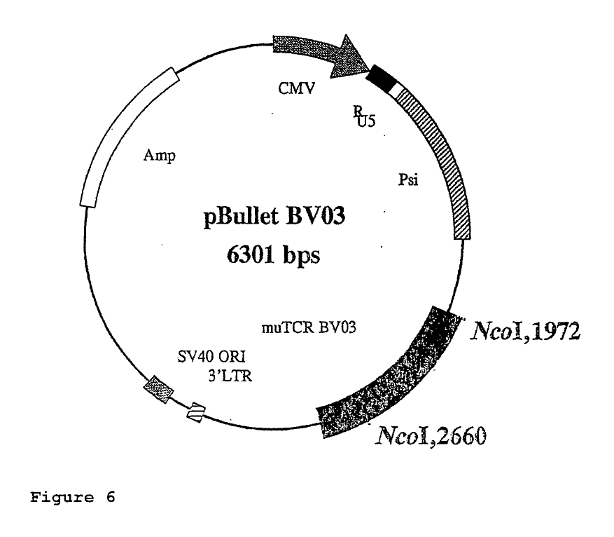

FIG. 6: Depiction of the functional Wt muTCR Vβ3 cloned into the retroviral pBullet vector.

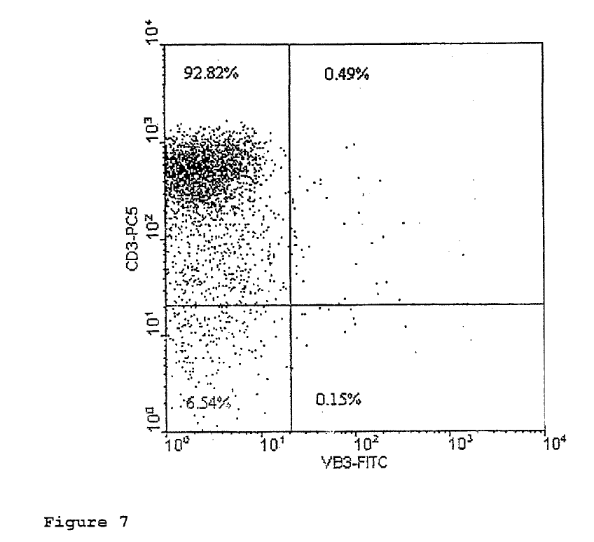

FIG. 7: Result of the flow-cytometric measurement of the PBMCs tranduced with the empty pBullet vector. No transgene (Vβ3) was detected.

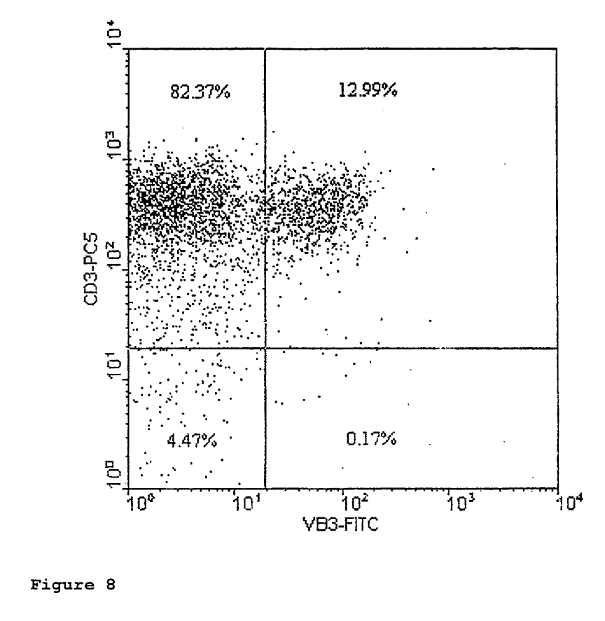

FIG. 8: Depiction of expression of the transgene Vβ3 as marker for reconstitution of mu-TCR expression on human PBMC, which was detected by flow cytometry. As expected, expression can be shown only for cells which additionally express the CD3 complex.

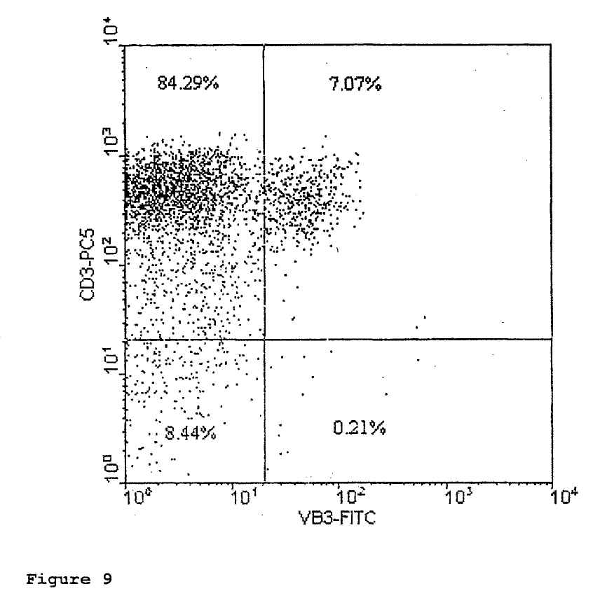

FIG. 9: Depiction of expression of the combination Vα13Vβ3, similar to FIG. 8, detection of the transgene Vβ3 suggests reconstitution of murine TCR expression on human PBMC.

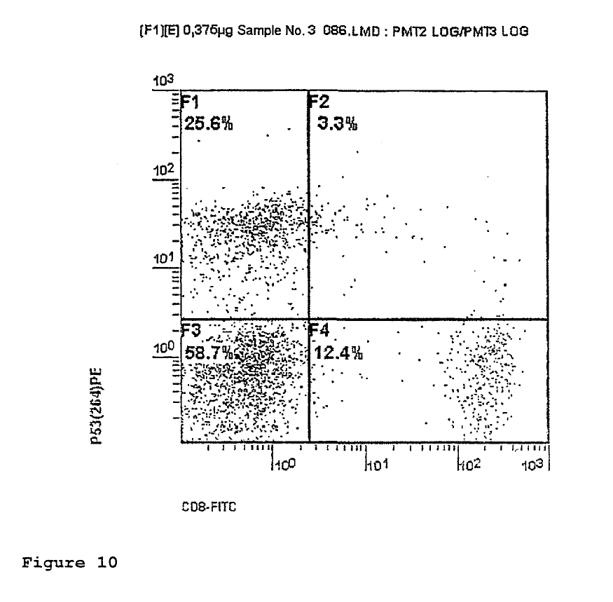

FIG. 10: Flow-cytometric depiction of human PBMCs transduced with Vα3Vβ3; stainability of HLA-A2.1-p53(264-272)-PE tetramer is shown, which indicates expression of a p53(264-272)-specific and HLA-A2.1-restricted TCR on the human PBMC.

SEQ ID No. 1: “Vα3”: productive, functional murine α chain (muvα-mucα); (see FIG. 2);

SEQ ID No. 2: “Vα13”: productive murine a chain (muvα-mucα); (see FIG. 2);

SEQ ID No. 3: “Vβ 1”: non productive, non functional murine β chain (muvβ-mucβ); (see FIG. 2);

SEQ ID No. 4: “Vβ 3”: productive, functional murine β chain (muvβ-mucβ); (see FIG. 2);

SEQ ID No. 5: “Vβ 3Cb0”: splicing variant of Vβ 3 with Cb0 insertion upstream of Cb1; (see FIG. 2);

SEQ ID No. 6: Primer GSP-1 (rev_R_a_SP1)

SEQ ID No. 7: Primer GSP-2 (rev_R_a_SP2)

SEQ ID No. 8: Primer GSP-3 (rev₁₃ Asc₁₃ aTCR_c1.2)

SEQ ID No. 9: Primer GSP-4 (rev_Asc_bTCR_c2)

SEQ ID No. 10: Primer GSP-5 (rev_bTCR_c4)

SEQ ID No. 11: Primer GSP-6 (rev_Asc_bTCR_c6)

SEQ ID No. 12: Vα3: Primer Forward (for Va3-NcoI_{—}1)

SEQ ID No. 13: Vβ3(Cβ0): Primer Forward (for_Vb3-NcoI_{—}1)

SEQ ID No. 14: Va13: Primer Reverse (for_Va13-NcoI_{—}1)

### EXAMPLES

Cytosolic mRNA was prepared using the commercially available QIAprep Miniprep (QIAGEN, Hilden, Germany) according to the manufacturer's protocol. The 5′RACE-PCR was carried out using the commercially available RACE PCR Kit (Roche Molecular Diagnostics) according to the manufacturer's protocol. As an alternative to reverse transcription within the RACE-PCR protocol, reverse transcription was carried out using displayTHERMO-RT (Display Systems Biotech, Vista, Calif., USA). For cloning into vectors pCR®2.1-TOPO® and pCR®XL-TOPO®, the corresponding kits (Invitrogen, Netherlands) according to the manufacturer's protocol were used. The cytotoxicity assays were carried out according to the method described in Theobald et al. (“Targeting p53 as a general tumor antigen”, 1995, Proc. Natl. Acad. Sci. USA 92, 11993-11997).

1. Cloning of the α-TCR Chains

After extracting the mRNA from a p53.264-272-specific, HLA-A2.1-restricted mouse CTL clone, the full length α-TCR chain was isolated via 5′-RACE-PCR (Boehringer Mannheim, Germany) by means of the self-designed gene-specific primers (SEQ ID No. 6 to SEQ ID No. 14). The specificity was increased by preparing the DNA intermediate (approx. 1100 bp) from an agarose gel prior to the second PCR (nested PCR). The products of approximately 1000 bp in length were subsequently cloned into the pCR®-XL-TOPO® vector system according to the manufacturer's instructions and sequenced.

FIG. 1 depicts diagrammatically the orientation of the primers and the cloning of the alpha chain. The gene-specific primers for amplification of the entire codogenic region of the TCR a chain were chosen so as to pair in the 3′-noncodogenic region (UTR). The gene-specific primer GSP-3 (SEQ ID No.8) which ultimately pairs on the stop codon introduces artificially an AscI site via its 5′-protruding end. The sequences of the gene-specific primers were determined by comparing published mouse TCR α-chain sequences and selection of suitable regions.

2. Cloning of the truncated TCR β chains

Cloning of the TCR β chain was carried out as for the a chain, but it was not possible here to use any gene-specific primers pairing outside the codogenic region, since various genes of the constant domain of the β chain exist. Therefore a 3′-truncated product had to be generated, making again use of the 5′-RACE PCR, and the product was sequenced. The product of the first PCR gave no distinct band in gel electrophoresis but was nevertheless extracted from the gel and supplied to the nested PCR. The resulting double band was then cloned into the TOPO® vector system (Invitrogen).

3. Analysis of the TCR chain sequences

Five different TCR chain-encoding sequences were distinguished by sequencing the PCR products:

1) Vα3: productive TCR α chain, functional (SEQ ID No. 1);

2) Vα13: productive TCR cc chain (SEQ ID No. 2);

3) Vβ1: non productive β chain due to faulty rearrangement (Vβ1->D beta: frame shift), non functional (SEQ ID No. 3);

4) Vβ3: productive β chain, functional (SEQ ID No. 4); and

5) Vβ3Cβ0: splicing variant of Vβ3 with Cβ0 insertion upstream of Cβ1 (SEQ ID No. 5).

4. Cloning of the Productive Chains Into the Retroviral pBullet Vector System

It was possible to derive from the sequences obtained primers for each chain, which pair in the 5′ region. These were modified in such a way (see SEQ ID Nos. 12-14) that it was possible to introduce via a PCR and an NcoI site (CCATGG) around the start codon ATG, thereby modifying, in the case of the α chains, the second base triplet and thus the second amino acid.

For cloning into the retroviral pBullet vector, first mRNA was again reversely transcribed (displayTHERMO-RT, cf. p. 20), this time, however, by using an oligo-dT primer (displayTHERMO-RT, cf. p. 20) which paired in the poly-A tail of the RNA, thereby producing a reverse transcript (single-stranded cDNA) of the entire RNA, which served as template in a subsequent PCR.

4.1 Cloning of Vα3/13

The TCR α chains were cloned by the above-described reverse transcription and PCR in which the flanking NcoI and SalI sites were introduced. pBullet and insert were NcoI and SalI-digested and the insert (Vα3/Vα13) was ligated by standard methods. After transformation of competent bacteria, positive clones were sequenced. An error-free Vα13 clone was chosen for further experiments. Since the yield of usable clones was low for Vβ3 and since one of these was acceptable apart from an error in the stop codon, the latter clone was again sub-cloned into an existing plasmid via an NcoI exchange cloning in order to reconstitute the stop codon, the 3′-flanking “site” in this case being BamHI-specific.

4.2 Cloning of Vβ3

The β chain (Vβ3) was cloned by cloning, after PCR, the coding nucleic acid once again into PCR®XL-TOPO® so as to be subcloned from there into the pBullet vector. For this purpose, a suitable clone was selected and first linearized by an AscI digest. Similarly, the empty pBullet vector was linearized by an XhoI digest. This was followed by filling in both cut “sticky” ends by T4 DNA polymerase in the presence of dNTPs to give “blunt” ends. Thereafter, a partial NcoI digest of the Vβ3 clone was carried out, since wild-type TCRs have an internal NcoI site which must not be cut in that digest, and, at the same time, the empty vector was completely digested with NcoI. After gel electrophoresis and extraction of the NcoI-full length Vβ3-blunt and the blunt-pBullet-empty-NcoI fragment, the insert and the vector were ligated. Bacteria clones transformed with the ligation product were sequenced.

5. Transduction of Human PBMCs

After establishing full-length TCR constructs inserted into the retroviral pBullet vector, these plasmids were amplified and prepared in bacteria cultures by methods known to the skilled worker. Said bacteria cultures were transfected in combination with the plasmids coding for the structural proteins gag, pol and GALV-env via Ca₃(PO₄)₂₋ transfection into the embryonal kidney cell line 293T. The following combinations were transfected:

1—pBullet+pHIT60+pCOLT-GaLV

2—pBullet AV03+pBullet BV03+pHIT60+PCOLT-GALV

3—pBullet AV13+pBullet BV03+pHIT60+pCOLT-GALV

This transient transfection (no introduced selection markers) resulted after about 24 h in the production of GALV-pseudotyped retroviral particles which, by coculturing the virus-producing 293T cells (irradiated with 2500 rad) with OKT-3(α-hu CD3)-activated HLA-A2-positive PBMCs of healthy donors, were utilized for infecting said PBMCs. The transduction efficiency was evaluated by flow cytometry approximately one week after three-day cocultivation and expansion and, after further expansion, it was possible to assay lytic reactivity.

6. FACS Analysis of Transduced PBMCs

The transduction efficiency was evaluated by measuring cells having the TCR-chain combination Vα3Vβ3, Vα13Vβ3 and also those which only with the pBullet expression vector which did not contain a transgene by using the flow cytometry technique. For this purpose, 10⁶ cells were stained according to the manufacturer's instructions with 1 μg of anti-muTCR Vβ3 antibody (BD Pharmingen, Heidelberg) and anti-huCD3 antibody at room temperature for 30 minutes and measured by flow cytometry. FIGS. 8 and 9 show that both the cells having the combination of Vα3Vβ3 and those having that of Vα13Vβ3 were positive stainable for CD3 and muTCR Vβ3, indicating membrane-bound expression of the β-chain transgene. In contrast, the negative control pBullet without transgene was non-positive for the Vβ3 surface transgene.

In order to further determine by flow cytometry the reconstitution of antigen specificity, 10⁵ cells were stained with 0.375 μg of A2-p53(264-272)-PE-tetramer (60 minutes on ice) and anti-huCD8-FITC. (15 minutes on ice) and measured by flow cytometry. The staining of previously muTCR Vβ3-positive, FACS-sorted PBMCs transduced with Vα3Vβ3 combination is shown by way of example (see FIG. 10).

7. Cytolytic Activity of Transduced PBMCs

The lytic reactivity of retroviral transduced human PBMC was evaluated by cytotoxicity assays. The transduced PBMCs were assayed in a standard chromium released assay, using firstly peptide-loaded T2 cells and secondly the p53 defect mutant Saos-2 and its mut (143 V->A) p53 transfectant Saos-2/143. The target cells all of which had the HLA-A2.1 phenotype were additionally admixed with a twenty-fold excess of K562 cells not labeled as chromium which served as “cold target” selectively as NK-cell target cells and thus reduced the unspecific NK-cell-mediated lysis of tumor cells. The ratio used of effector:target cells (E:T) was 30.

A p53 polypeptide-specific reaction of the PBMCs transduced with the Vα3Vβ3 combination can be detected. Moreover p53-derived peptide which is endogenously processed and presented in the context of HLA-A2.1 is detected (Saos-2/143), this being regarded as necessary prerequisite for the lysis of p53-overexpressing tumor cells. A lysis of the negative control Saos-2 could not be demonstrated for any effector cells. Table 1 below depicts by way of example the specific lysis measured of various target cells. It was furthermore possible to detect a specific lysis of malignant cell-lines (not shown).

TABLE 1

| Target cell\effector | pBullet | Vα3Vβ3 | Vα13Vβ3 |
|---|---|---|---|
| T2 + FluMI | 9 | 20 | 19 |
| T2 + p53.264-272 | 6 | 88 | 5 |
| Saos-2 | 4 | 6 | 7 |
| Saos-2/143 | 1 | 31 | 4 |

Data in % specific lysis, ratio E:T=30:1

1. A polypeptide of a murine α/β T-cell receptor mediating a p53 protein-specific T-cell response according to SEQ ID No. 1 to SEQ ID No. 5 or functional variants or parts thereof or nucleic acids encoding this, functional variants or parts thereof.

2. A fusion protein, comprising a polypeptide as claimed in claim 1 or functional variants or parts thereof or nucleic acids encoding this, functional variants or parts thereof.

3. The fusion protein as claimed in claim 2, characterized in that it comprises the ζ-region of CD3 and/or CD8, CD 16 or parts thereof, in particular the ζ-region of human CD3 and/or CD8, CD16 or parts thereof.

4. The fusion protein as claimed in claim 2 or 3, furthermore comprising a flexible linker, in particular a linker of the amino acid sequence (GGGGS)₃.

5. The fusion protein as claimed in claim 2, characterized in that it is a chimeric, at least partially humanized fusion protein.

6. The fusion protein as claimed in one of claims 2 to 5, characterized in that it is a single-chain or double-chain T-cell receptor.

7. An α- or β-chain of a T-cell receptor, which comprises the antigen recognition sequence of an antibody specific for the amino acids 264-272 of the protein p53 or of a complex of p53 264-272 and HLA-A2.

8. The polypeptide as claimed in one of claims 1 to 7, characterized in that the polypeptide has been synthetically prepared.

9. The nucleic acid as claimed in claim 1 or 2, characterized in that it is a DNA or RNA, preferably a DNA, in particular a double-stranded DNA having a length of at least 8 nucleotides, preferably having at least 18 nucleotides, in particular having at least 24 nucleotides.

10. The nucleic acid as claimed in one of claims 1, 2 or 9, characterized in that the sequence of the nucleic acid contains at least one intron and/or one polyA sequence.

11. The nucleic acid as claimed in one of claims 1, 2, 9 or 10 in the form of its complementary “antisense” sequence.

12. The nucleic acid as claimed in one of claims 1, 2 or 9 to 11, characterized in that the nucleic acid has been synthetically prepared.

13. A vector, preferably in the form of a plasmid, shuttle vector, phagemid, cosmid, expression vector, retroviral vector, adenoviral vector or particle and/or vector active in gene therapy, comprising a nucleic acid as claimed in one of claims 1, 2 or 9 to 12.

14. A host cell, transfected with a vector or infected or transduced with a particle as claimed in claim 13.

15. The host cell as claimed in claim 14, characterized in that it is a T-cell or a T-precursor cell or a stem cell.

16. The host cell as claimed in claim 14 or 15, characterized in that it expresses a polypeptide or fusion protein according to one of claims 1 to 8 on its surface.

17. A process for the identification of p53 protein-specific antigens, characterized in that p53-presenting tumor cells or fractions thereof are combined with a host cell as claimed in claim 16 under conditions in which the tumor cells or fractions thereof are only lyzed if the tumor presents the p53 protein-specific antigen for which the presented polypeptide or fusion protein is specific.

18. A process for the preparation of an antibody, preferably a polyclonal or monoclonal antibody, directed against a polypeptide, fusion protein or a nucleic acid as claimed in one of claims 1 to 12 for the diagnosis, treatment and/or monitoring of the treatment of diseases associated with p53 protein and/or for the identification of pharmacologically active substances, characterized in that an antibody-producing organism is immunized with a polypeptide or functional equivalents thereof or parts thereof having at least 6 amino acids, preferably having at least 8 amino acids, in particular having at least 12 amino acids as claimed in one of claims 1 to 8 or with nucleic acids encoding this.

19. An antibody, prepared as claimed in claim 18, characterized in that it is directed against a polypeptide as claimed in one of claims 1 to 8.

20. A recombinant antibody, characterized in that it comprises the antigen recognition sequence of the α- or β-chain of a T-cell receptor specific for the amino acids 264-272 of the protein p53 or of a complex of p53 264-272 and HLA-A2.

21. A process for the production of a medicament for the treatment of diseases associated with p53 protein, characterized in that at least one nucleic acid, at least one polypeptide, at least one host cell or at least one antibody as claimed in one of the aforementioned claims is combined, together with suitable additives and excipients.

22. A medicament for the treatment of diseases associated with p53 protein, characterized in that it contains at least one nucleic acid, at least one polypeptide, at least one host cell or at least one antibody as claimed in one of the aforementioned claims, if appropriate together with suitable additives and excipients.

23. The use of a medicament as claimed in claim 22 for the treatment of diseases associated with p53 protein.

24. A process for the production of a test for the discovery of functional interactors in connection with diseases associated with p53 protein, characterized in that at least one nucleic acid, at least one polypeptide or at least one antibody as claimed in one of the aforementioned claims is combined, together with suitable additives and excipients.

25. A test for the identification of functional interactors in connection with diseases associated with p53 protein, characterized in that it contains at least one nucleic acid, at least one polypeptide or at least one antibody as claimed in one of the aforementioned claims, if appropriate together with suitable additives and excipients.
